(19) United States
(12) Patent Application Publication
(10) Pub. No.: US 2026/0215861 A1
(43) Pub. Date: Jul. 30, 2026

(54) Controlling a Robotic System Actuator for Driving an Elongated Flexible Medical Instrument

(71) Applicant: ROBOCATH, ROUEN (FR)

(72) Inventor: Camille APAMON, RENNES (FR)

(21) Appl. No.: 19/142,751

(22) PCT Filed: Dec. 26, 2023
(86) PCT No.: PCT/FR2023/052126

§ 371 Date: Aug. 8, 2025

(30) Foreign Application Priority Data
Dec. 27, 2022 (FR) FR2214551

(51) Int. Cl. A61B 34/30; A61B 90/00

(52) U.S. Cl. CPC A61B 34/30; A61B 90/06; A61B 2034/301; A61B 2090/064

(57) ABSTRACT

This method (200) is intended for controlling an actuator connected kinematically to a drive member capable of coming into contact with an elongate flexible medical instrument so as to control the movement of said drive member along an axis. It comprises measuring (206) a force exerted by the medical instrument on the drive member along the axis, transmitting (214) to the actuator a primary control signal suitable for causing a speed of movement of the drive member along the axis to converge towards a predetermined speed setpoint as long as the measured force is below a predetermined threshold, and transmitting (216) to the actuator a secondary control signal suitable for causing the measured force to converge towards a predetermined force setpoint when the measured force exceeds said threshold.

### CROSS REFERENCE TO RELATED APPLICATIONS

This application is the 35 U.S.C. 371 National Stage Application of PCT Application No. PCT/FR 2023/052126, filed Dec. 26, 2023, which application claims the benefit of French Application No. FR 2214551, filed Dec. 27, 2022, both of which are hereby incorporated by reference herein in their entireties.

### FIELD OF THE INVENTION

This invention relates to robotic systems for driving elongate flexible medical instruments such as guides, catheters, etc. used in particular in angioplasty procedures. It more specifically relates to the control of the actuators of such a robotic system.

### TECHNICAL BACKGROUND

Angioplasty with manual insertion of an elongate flexible medical instrument such as a catheter or a guide into a patient is a relatively conventional medical procedure. However, since this procedure is followed up with X-ray angiography, the practitioner in charge of this procedure is exposed to significant radiation if he performs such an operation on a large number of patients.

To reduce the risk to the practitioner, provision has been made for robotically controlling such an insertion by means of robotic systems comprising clamping members, displaced by actuators, which can move closer to or apart from one another to respectively clamp or release said medical instrument, said clamping members, once moved closer to one another in such a way as to clamp the medical instrument, being able to carry out:
a synchronous longitudinal translation to move the medical instrument forward or backward, and/or
opposite transverse translations to rotate the medical instrument about its axis of elongation.

Such a robot is known for example from FR 3 044 541.

The clamping force applied by the clamping members of these robotic systems must meet two antagonistic requirements. Firstly, it must be sufficient to allow the driving of the medical instrument without slipping. Secondly, it must not be too high to avoid the ovalization of the medical instrument, i.e. the modification of the cross section of said medical instrument which would change from being circular when new to being oval or elliptic under the effect of the gripping force. Such an ovalization is specifically a hindrance during the driving of the medical instrument in rotation since it can oppose the correct rotation of the medical instrument, as described in WO 2022/219165.

Generally, to displace the clamping members from their apart position to their closer position, the actuators are controlled by means of a control law aiming to displace the members into a given position, according to the type of the medical instrument handled, which must have been previously inputted by the robot operator. The clamping members are then kept in this position as long as they are in a phase during which they must clamp the medical instrument. This does however often lead to an ovalization of the medical instrument.

To solve this problem, provision has been made in WO 2022/219165 for equipping these robotic systems with sensors that measure the clamping force and with a regulator which keeps the clamping force between a minimum threshold and a maximum threshold. Thus, when the actuators are in the closer position, their clamping force is regulated.

Nonetheless, this solution does not provide complete satisfaction. Specifically, WO 2022/219165 does not detail how this controlling of the clamping force of the clamping members is coordinated with the control of the displacement of the clamping members from their apart position to their closer position. However, the inventors observed that it was difficult to control this displacement of the clamping members such as to reconcile observance of the maximum threshold of the clamping force and speed of movement of the clamping members.

### SUMMARY OF THE INVENTION

One aim of the invention is to allow a rapid displacement, along a direction of displacement, of a driving member intended to come into contact with an elongate flexible medical instrument while avoiding the force exerted by the driving member on the elongate flexible medical instrument along the direction of displacement exceeding a given threshold. Other aims are to avoid damage to the medical instrument and to allow the driving of the medical instrument by the driving member without slipping.

For this purpose, the invention has the subject, according to a first aspect, of a method for controlling a robotic system for driving, in translation and/or in rotation, an elongate flexible medical instrument, the robotic system comprising a driving member able to come into contact with the elongate flexible medical instrument and kinematically linked to the actuator such that the latter controls the movement of said driving member along a so-called main axis, the control method being implemented by a data processing unit and comprising the following steps:
measuring a speed of movement of the driving member along the main axis,
measuring a force exerted by the elongate flexible medical instrument on the driving member along the main axis, and
displacing the driving member along the main axis, in a first direction, with:
   transmitting, to the actuator, a primary control signal suitable for making the measured speed of movement converge on a given speed setpoint as long as the measured force is below a non-zero given force threshold, and
   transmitting, to the actuator, a secondary control signal suitable for making the measured force converge on a given force setpoint when the measured force exceeds said force threshold.

According to particular embodiments of the invention, the control method also has one or more of the following features, taken in isolation or according to any technically possible combination(s):
the main axis consists in a clamping axis of the elongate flexible medical instrument, the first direction being oriented toward the elongate flexible medical instrument;
the method comprises the following additional steps:
   measuring a position of the driving member along the main axis, and
   displacing the driving member along the main axis in a second direction opposite to the first direction, with transmitting to the actuator a tertiary control signal suitable for making the measured position converge on a given position setpoint;
the method comprises, between the steps of displacing in the first direction and in the second direction, an additional step of immobilizing the driving member along the main axis during which there is transmitted to the actuator a quaternary control signal suitable for keeping the measured force substantially equal to the force setpoint;
the method comprises, during the step of immobilizing the driving member along the main axis, displacing the driving member along at least one axis orthogonal to the main axis;
the steps of displacing in the first direction and in the second direction are implemented one after the other, in a cyclic repeated manner;
the measured position is deduced from a current position of the actuator;
the method comprises an additional step of measuring a position of the driving member along the main axis, the speed setpoint having a first value as long as the measured position is below a given position threshold and a second value, less than the first value, when the measured position is above said position threshold;
the position threshold is such that when the measured position is equal to said position threshold the driving member is not in contact with the elongate flexible medical instrument;
the force setpoint is greater than the force threshold;
the measured displacement speed is deduced from a current speed of the actuator;
the measured force is deduced from a current supply power of the actuator;
the measured force is measured directly by a stress sensor;
the elongate flexible medical instrument consists of a catheter or a catheter guide;
the force exerted by the driving member on the elongate flexible medical instrument does not exceed a given limit, preferably less than or equal to 30 N; and
the method does not comprise any step of determining the distance between the driving member and the elongate flexible medical instrument.

Another subject of the invention, according to a second aspect, is a robotic system for driving an elongate flexible medical instrument, said robotic system comprising a frame, a driving member able to come into contact with the elongate flexible medical instrument, an actuator kinematically linked to the driving member such as to control the movement of said driving member in relation to the frame along a main axis, and a data processing unit for implementing a method according to the first aspect.

The invention also has the subject, according to a third aspect, of a computer program product comprising code instructions for implementing a control method according to the first aspect when said computer program product is executed by a processor of a data processing unit of a robotic system for driving an elongate flexible medical instrument.

Finally the invention has the subject, according to a fourth aspect, of a storage means readable by an item of computer equipment on which is recorded a computer program product according to the third aspect.

### BRIEF DESCRIPTION OF THE FIGURES

Other features and advantages of the invention will become apparent on reading the following description, given solely by way of example and with reference to the appended drawings, wherein:

FIG. 1 is a schematic side view of an example of an angioplasty installation comprising a robotic system in accordance with an embodiment of the invention,

FIG. 2 is a schematic perspective view of the robotic system of the installation of FIG. 1,

FIGS. 3A to 3E schematically represent several successive steps of rotational driving of an elongate flexible medical instrument by the robotic system of FIG. 2,

FIGS. 4A to 4G schematically represent several successive steps of translational driving of an elongate flexible medical instrument by the robotic system of FIG. 2,

FIG. 5 is a functional diagram of a control unit of the robotic system of FIG. 2,

FIG. 6 is a diagram illustrating a method for controlling actuators of the robotic system of FIG. 2, and

FIG. 7 is a diagram illustrating the variation over time of various parameters of the robotic system of FIG. 2 when the control method of FIG. 6 is applied.

### DETAILED DESCRIPTION OF AN EXEMPLARY EMBODIMENT

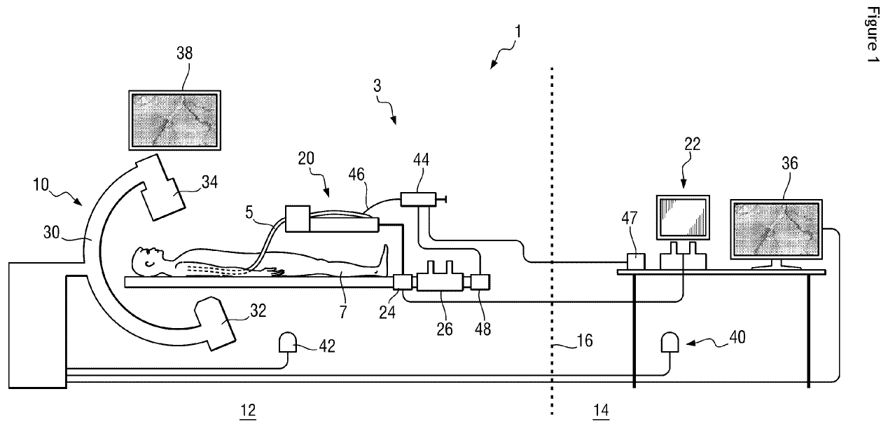

The angioplasty installation 1 shown on FIG. 1 comprises a robot 3 for introducing an elongate flexible medical instrument 5 into an anatomical duct of a patient 7, typically into a blood vessel of said patient 7. It also comprises an angiography system 10 to track the displacement of the medical instrument 5 inside the body of the patient 7.

The angioplasty installation 1 is here distributed between an operating room 12 and a control room 14. In an embodiment, this control room 14 is near the operating room 12 and is for example separated therefrom by a single wall 16 opaque to X rays. In another embodiment, the control room 14 is distant from the operating room 12. In a variant (not shown), the angioplasty installation 1 is entirely disposed in the single operating room 12.

The robot 3 comprises a robotic system 20 for driving the medical instrument 5, placed in the operating room 12 near the patient 7.

The robot 3 also comprises a control station 22 for the controlling of the robotic system 20 by an operator. Here, this control station 22 is a remote control station placed in the control room 14 and communicating with the robotic system 20 via a communication unit 24 connected to the robotic system 20.

In the example shown, the robot 3 also comprises a local control unit 26, disposed in the operating room 12, for the controlling of the robotic system 20 by an operator directly from the operating room 12.

The angiography system 10 comprises a medical imaging device 30, particularly an X-ray imaging device, including a source 32 and a detector 14 disposed on either side of the patient 7, optionally movable with respect to the patient 7.

The angiography system 10 also comprises at least one screen 36, 38 communicating with the imaging device 30 for the real-time display of the images captured by the imaging device 30. Here, the screens 36, 38 comprise a remote screen 36 installed in the control room 14 and a local screen 38 installed in the operating room 12. In a variant (not shown), the angiography system 10 also comprises the remote screen 36 or the local screen 38.

The angiography system 10 further comprises at least one controller 40, 42 communicating with the imaging device 30 to control the taking of images by the imaging device 30. Here, the controllers 40, 42 comprise a remote controller 40 installed in the control room 14 and a local controller 42 installed in the operating room 12. In a variant (not shown), the angiography system 10 comprises only the remote controller 40 or the local controller 42.

The angiography system 10 finally comprises an injector 44 of contrast agent for the injection, into the medical instrument 5, of a contrast agent facilitating the imaging of said medical instrument 5, a connector 46 connecting the injector 44 to the medical instrument 5 to guide the contrast agent from the injector 44 all the way into the medical instrument 5, and at least one controller 47, 48 to control the injector 44. Here, the controllers 47, 48 comprise a remote controller 47 installed in the control room 14 and a local controller 48 installed in the operating room 12. In a variant (not shown), the angiography system 10 comprises only the remote controller 47 or the local controller 48.

The elongate flexible medical instrument 5 is elongate along an axis of elongation. It consists of a medical instrument able to be inserted into an anatomical duct, typically a blood vessel, of the patient 7, and to be displaced in said anatomical duct through a Desilet forming an access opening in the patient 7. This elongate flexible medical instrument 5 typically consists of a catheter or of a guide for a catheter.

A catheter, in a known manner, consists of a flexible and elongate tube, which is generally hollow over a portion near the patient 7, or even over the entirety of its length. Optionally, the catheter is equipped, at its distal end (opposite the robot 3), with a medical tool such as balloon, a stent, etc.

A guide is, in a known manner, a medical instrument configured to guide the catheter all the way to an implantation site in the body of the patient 7. For this purpose, the guide generally consists of a cylinder of smaller transverse diameter than that of the catheter such that the catheter can be placed around the guide and slide along the guide under the effect of a force initiated by the robot 3 or by an operator until its free end reaches the desired implantation site. Optionally, the guide includes a curved end, such as to facilitate its navigation through the bloodstream of the patient 7.

To displace the elongate flexible medical instrument 5 inside the body of the patient 7, it is desirable to be able to translate said instrument 5 along its axis of elongation and to be able to rotate it about said axis of elongation. The reader may refer to the document FR 3 044 541 for more details as to the usefulness of these movements.

The robotic system 20 is configured to drive the elongate flexible medical instrument 5 so as to impart to it at least one of these movements, here both.

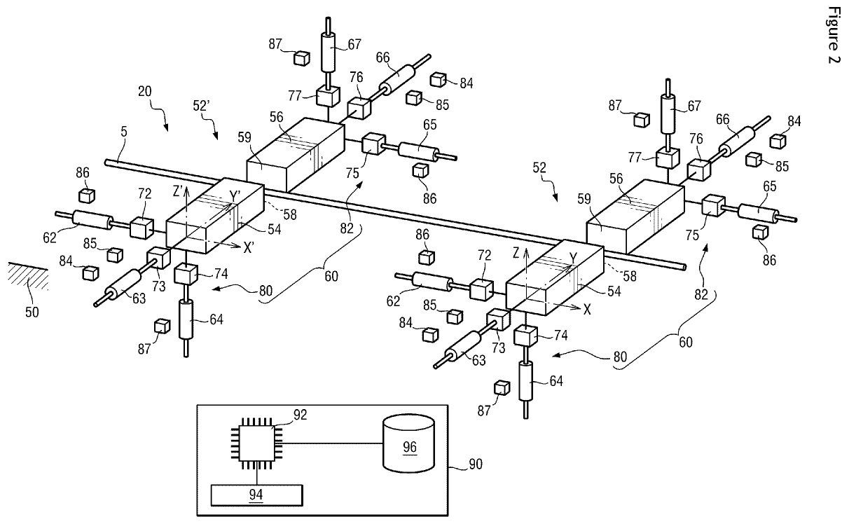

For this purpose, the robotic system 20 comprises, with reference to FIG. 2, a frame 50 and at least one, here two, driving modules 52, 52′ each configured to drive the medical instrument 5 in relation to the frame 50:
in translation along an axis X, X′ of extension of the medical instrument 5 at said driving module 52, 52′ here described as a longitudinal axis, and
in rotation about said longitudinal axis X, X′.

Note that said longitudinal axis X, X′ is usually different from the axis of extension of the medical instrument 5 at its distal end; nonetheless, a translation and/or a rotation of the medical instrument 5 along/about the longitudinal axis X, X′, and therefore along/about its axis of elongation at the robotic system 20 will drive a translation and/or a rotation of the medical instrument 5, respectively, along/about its axis of elongation at its distal end.

In the example shown, each driving module 52, 52′ comprises a pair of driving members 54, 56 together forming a clamp configured to grip and displace the medical instrument 5 in relation to the frame 50.

For this purpose, at least one of said driving members 54, 56, here each of the driving members 54, 56, is mounted translationally movably in relation to the frame 50 along a clamping axis Y, Y′ orthogonal to the longitudinal axis X, X′ and substantially secant of the axis of the medical instrument 5. The driving members 54, 56 are movable with respect to one another along this clamping axis Y, Y′ between a further position, in which the driving members 54, 56 are distant from one another, and a closer position in which the driving members 54, 56 are close to one another.

Each driving member 54, 56 of one pair is moreover translationally movable in relation to the frame 50 along at least one other axis substantially orthogonal to the clamping axis Y, Y′. Each driving member 54, 56 of a pair is thus translationally movable in relation to the frame 50 along at least one of the following axes:
the longitudinal axis X, X′, and
a transverse axis Z, Z′ substantially orthogonal to the clamping axes Y, Y′ and longitudinal axes X, X′.

The two driving members 54, 56 of each driving module 52, 52′ in particular have the same degrees of freedom along the longitudinal axis X, X′, i.e. for each driving module 52, 52′ of which one of the driving members 54, 56 is translationally movable along the longitudinal axis X, X′, the other driving member 54, 56 of said driving module 52, 52′ is also translationally movable along said longitudinal axis X, X′. Advantageously, the two driving members 54, 56 of each driving member 52, 52′ also have the same degrees of freedom along the transverse axis Z, Z′.

Here, each driving member 54, 56 of each driving module 52, 52′ is translationally movable in relation to the frame 50 along each of these longitudinal X, X′ and transverse Z, Z′ axes.

Preferably, the driving modules 52, 52′ are arranged such that the longitudinal axes X, X′ are, as shown, substantially colinear. In the remainder of the text, one will thus, for the sake of simplicity, simply refer to the longitudinal axis X, the clamping axis Y and the transverse axis Z.

Each driving member 54, 56 delimits a driving surface, respectively 58, 59, configured to be distant from the medical instrument 5 when the driving members 54, 56 are in the further position and in contact with the medical instrument 5 when the driving members 54, 56 are in the closer position. Said driving surfaces 54, 56 face one another and are spaced apart from one another along the axis Y. Each driving surface 58, 59 in particular has a normal substantially parallel to the axis Y.

Each driving member 54, 56 is typically formed of a pad holder (not shown) and a removable pad (not shown) mounted on the pad holder and delimiting the driving surface 58, 59. The driving surface 58, 59 in contact with the medical instrument 5 can thus be changed each time the robot 3 is used, which makes it possible to preserve the sterility of the medical instrument 5.

Each driving module 52, 52′ also comprises a driving device 60 to control the displacement of the driving members 54, 56 of said module 52, 52′ along the axis Y and, where applicable, the axis X and/or Z. This driving device 60 comprises at least one actuator 62, 63, 64, 65, 66, 67 and, for the or each actuator 62, 63, 64, 65, 66, 67, a kinematic linkage, respectively 72, 73, 74, 75, 76, 77 kinematically linking said actuator 62, 63, 64, 65, 66, 67 to at least one of the driving members 54, 56 such that this latter controls the displacement of said driving member 54, 56 along at least one of the axes X, Y, Z.

According to a possible variant, as shown, each kinematic linkage 72, 73, 74, 75, 76, 77 kinematically links an actuator 62, 63, 64, 65, 66, 67 to a single one of the driving members 54, 56. Each actuator 62, 63, 64, 65, 66, 67 thus controls the displacement of a single one of the driving members 54, 56. The driving device 60 is thus formed of two driving sub-devices 80, 82, each specific to one of the driving members 54, 56.

Here, each driving sub-device 80, 82 comprises three actuators, respectively 62, 63, 64 and 65, 66, 67. Each actuator 62, 63, 64, 65, 66, 67 contributes to the displacement of the driving member, respectively 54, 56, along at least one of the axes X, Y, Z. Advantageously, each actuator 62, 63, 64, 65, 66, 67 contributes to the displacement of the driving member, respectively 54, 56, along a single axis, specific to said actuator 62, 63, 64, 65, 66, 67, from among the axes X, Y, Z or, as default, contributes mostly along one axis, specific to said actuator, 62, 63, 64, 65, 66, 67, from among the axes X, Y, Z, i.e. the contribution of said actuator 62, 63, 64, 65, 66, 67 to the displacement of the driving member 54, 56 along said specific axis is large compared to the contribution of the actuator 62, 63, 64, 65, 66, 67 to the displacement of the driving members 54, 56 along each of the other axes. Thus, in the example shown:
the actuators 62, 65 contribute mostly or exclusively to the displacement of the driving members 54, 56 along the axis X,
the actuators 63, 66 contribute mostly or exclusively to the displacement of the driving members 54, 56 along the axis Y, and
the actuators 64, 67 contribute mostly or exclusively to the displacement of the driving members 54, 56 along the axis Z.

For this purpose, each driving sub-device 80, 82 typically consists of a driving device as described in WO 2022/144267, the contents of which are here incorporated by reference.

Each actuator 62, 63, 64, 65, 66, 67 consists for example in an electric motor comprising a rotor and a stator (not shown), the stator being fixed in relation to the frame 50 and the rotor forming the part of the actuator 62, 63, 64, 65, 66, 67 kinematically linked to the driving member 54, 56.

The robotic system 20 also comprises sensors 84, 85 for measuring a clamping force, a displacement speed and a position of the driving members 54, 56 of each module 52, 52′ along the axis Y. Here, the robotic system 20 additionally comprises sensors 87, 88 for also measuring a position of the driving members 54, 56 of each module 52, 52′ along each of the axes X and Z.

The sensors 84, 85, 87, 88 here are indirect sensors, i.e. they supply data representative of the clamping force, of the displacement speed along the axis Y and of the position of the driving members 54, 56 of each module 52, 52′ by indirect measurements, here by measurements on the actuators 62, 63, 64, 65, 66, 67. These indirect measurements are for example a measurement of the supply current of the actuators 62, 63, 64, 65, 66, 67 and a measurement of the position of the actuators 62, 63, 64, 65, 66, 67 (typically, in the case of electric motors, of the position of the rotor in relation to the stator). Specifically, transfer functions are known to exist linking:
the electrical power consumed by an actuator with the force exerted by a member driven by said actuator,
the speed of an actuator with the displacement speed of a member driven by said actuator, and
the position of an actuator with the position of a member driven by said actuator,

these transfer functions dependent on the kinematic linkage linking the actuator to the member it drives. Those skilled in the art will easily be able to find these transfer functions to deduce the clamping force, the displacement speed along the axis Y and the position of the driving members 54, 56 of each module 52, 52′ based on the measurements supplied by the sensors 84, 85, 87, 88.

In a variant (not shown), the sensors 84, 85, 87, 88 are direct sensors, i.e. they directly measure the clamping force, the displacement speed along the axis Y and the position of the driving members 54, 56 of each module 52, 52′.

The robotic system 20 further comprises a unit 90 for controlling the actuators 62, 63, 64, 65, 66, 67 of each drive module 52, 52′, suitable for transmitting to each of said actuators 62, 63, 64, 65, 66, 67 a control signal of this latter.

This control unit 90 is here embodied in the form of a data processing unit comprising a processor or CPU (Central Processing Unit) 92, a memory 94 of RAM (Random Access Memory) and/or ROM (Read Only Memory) type, and a storage module 96 of internal storage type.

The storage module 96 is for example of HDD (Hard Disk Drive) or SSD (Solid-State Drive) type, or of external storage support reader type, such as an SD (Secure Digital) card reader.

The processor 92 is configured to record data, or information, in the memory 94 or in the storage module 96 and/or read data recorded in the memory 94 or in the storage module 96.

The processor 92 is configured to execute instructions loaded into the memory 94, for example from the storage module 96. When the robotic system 20 is energized, the processor 92 is capable of reading instructions 94 from the memory and executing them. These instructions form a computer program causing the implementation, by the processor 92, of all or part of a method 200 for controlling the actuators 62, 63, 64, 65, 66, 67 which will be described further on. Thus, all or part of the method 200 can be implemented in software form by the execution of a set of instructions by a programmable machine, such as a DSP (Digital Signal Processor) or a microcontroller.

In a variant (not shown), the control unit 90 is embodied in the form of a data processing unit consisting, at least in part, of a machine or a dedicated component, such as an FPGA (Field-Programmable Gate Array) or an ASIC (Application-Specific Integrated Circuit), for implementing all or part of the method 200.

Note that, although shown here in the form of a single unit common to all the actuators 62, 63, 64, 65, 66, 67, the control unit 90 can in a variant be embodied in the form of several distributed units at each driving device 60 or at each sub-device 80, 82, or at each actuator 62, 63, 64, 65, 66, 67, said distributed units being then synchronized by means of a shared synchronization signal.

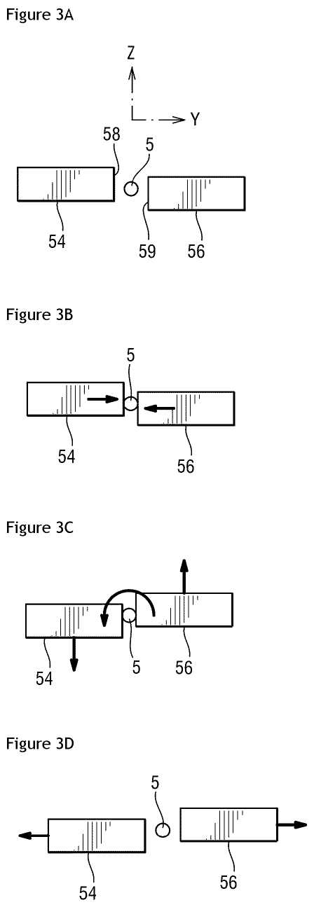

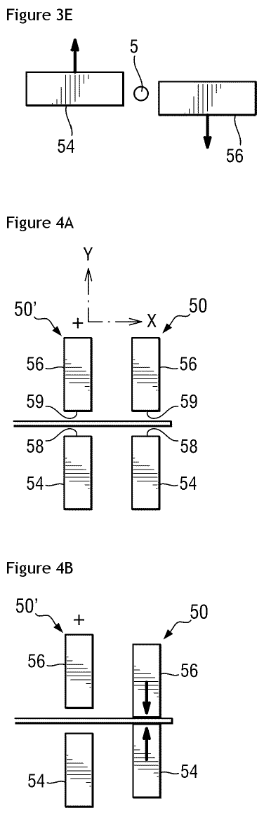

The control unit 90 is in particular configured to control the actuators 62, 63, 64, 65, 66, 67 so as to drive the rotation of the medical instrument 5 about its axis of elongation by simultaneous displacement, in the reverse direction, of the driving members 54, 56 of one and the same pair along the axis Z. For this purpose, the control unit 90 is typically configured to control the implementation of the following steps, illustrated on FIGS. 3A to 3E:
positioning of the driving members 54, 56 in the further position, their respective driving surfaces 58, 59 not being in contact with the medical instrument 5 (FIG. 3A);
bringing the driving members 54, 56 closer to one another by translating said members 54, 56 in opposite directions along the axis Y, until their respective driving surfaces 58, 59 clamp around the medical instrument 5 (FIG. 3B);
displacing the driving members 54, 56 in translation along the axis Z, in opposite directions to one another, such as to rotate the medical instrument 5 about its axis in one direction or the other, the driving surfaces 58, 59 keeping the medical instrument 5 clamped during this displacement (FIG. 3C);
moving the driving members 54, 56 away from the medical instrument 5 by translating said members 54, 56 in opposite directions along the axis Y, until a given position is reached in which the driving surfaces 58, 59 release the medical instrument 5 and then stop being in contact with the medical instrument 5 (FIG. 3D); and
repositioning the driving members 54, 56 by translating said members 54, 56 in opposite directions along the axis Z, their respective driving surfaces 58, 59 remaining distant from the medical instrument 5 such as to avoid driving the medical instrument 5 in rotation, until the driving members 54, 56 regain their original position (FIG. 3E).

The control unit 90 is in particular configured to control the cyclical repetition of these steps, in such a way as to allow a complete rotation of the medical instrument 5 in one direction as in the other.

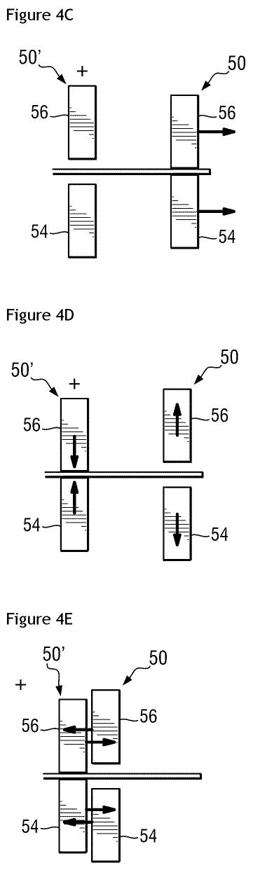

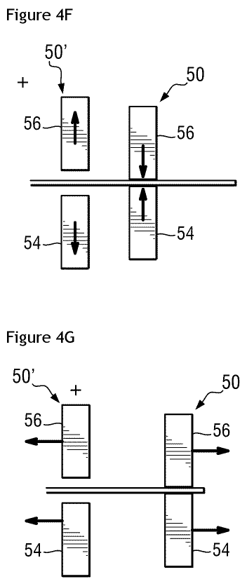

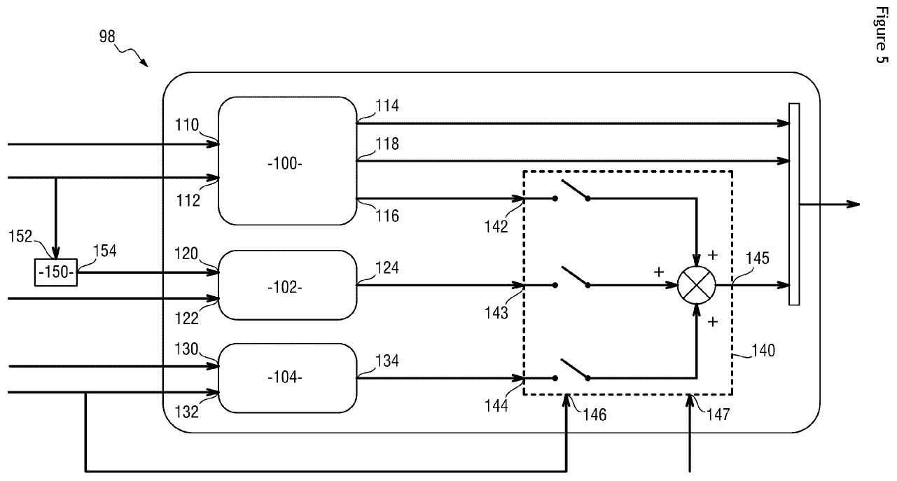

The control unit 90 is also configured to control the actuators 62, 63, 64, 65, 66, 67 such as to drive the translation of the medical instrument 5 along its axis by simultaneous displacement, in the same direction, of the driving members 54, 56 of one and the same pair along the axis X. For this purpose, the control unit 90 is typically configured to control the implementation of the following steps, illustrated on FIGS. 4A to 4G:
positioning the driving members 54, 56 of each driving module 50, 50′ in the further position, their respective driving surfaces 58, 59 not being in contact with the medical instrument 5 (FIG. 4A);
bringing the driving members 54, 56 of a first driving module 50 closer to one another by translating said members 54, 56 in opposite directions along the axis Y, until their respective driving surfaces 58, 59 clamp the medical instrument 5, the driving members 54, 56 of the second driving module 50′ remaining distant from the medical instrument 5 (FIG. 4B);
displacing the driving members 54, 56 of the first driving module 50 in translation along the axis X, synchronously and in one and the same first direction, such as to drive the medical instrument 5 in translation along its axis in said first direction, the driving surfaces 58, 59 of the first driving module 50 keeping the medical instrument 5 clamped during this displacement, while the driving members 54, 56 of the second driving module 50′ remain immovable and distant from the medical instrument 5 (FIG. 4C);
moving the driving members 54, 56 of the first driving module 50 away from the medical instrument 5 by translating said members 54, 56 in opposite directions along the axis Y, until a given position is reached in which the driving surfaces 58, 59 of said members 54, 56 release the medical instrument 5 and then stop being in contact with the medical instrument 5, and simultaneously bringing the driving members 54, 56 of the second driving module 50′ closer to one another by translating said members 54, 56 in opposite directions along the axis Y, until their respective driving surfaces 58, 59 clamp the medical instrument 5 (FIG. 4D);
displacing the driving members 54, 56 of the second driving module 50′in translation along the axis X, synchronously and in the first direction, such as to drive the medical instrument 5 in rotation along an axis in the first direction, the driving surfaces 58, 59 of the second driving module 50′ keeping the medical instrument 5 clamped during this displacement, and simultaneously displacing the driving members 54, 56 of the first driving module 50 in translation along the axis X, synchronously and in the second direction, their respective driving surfaces 58, 59 remaining away from the medical instrument 5 so as not to drive the medical instrument 5, until the driving members 54, 56 of the first driving module 50 regain their original position (FIG. 4E);
moving the driving members 54, 56 of the second driving module 50′away from the medical instrument 5 by translating said members 54, 56 in opposite directions along the axis Y, until a given position is reached in which the driving surfaces 58, 59 of said members 54, 56 release the medical instrument 5 and then stop being in contact with the medical instrument 5, and simultaneously bringing the driving members 54, 56 of the first driving module 50 closer to one another by translating said members 54, 56 in opposite directions along the axis Y, until their respective driving surfaces 58, 59 clamp the medical instrument 5 (FIG. 4F)
displacing the driving members 54, 56 of the first driving module 50 in translation along the axis X, synchronously and in the first direction, such as to drive the medical instrument 5 in translation along its axis in the first direction, the driving surfaces 58, 59 of the first driving module 50 keeping the medical instrument 5 clamped during this displacement, and simultaneously displacing the driving members 54, 56 of the second driving module 50′ in translation along the axis X, synchronously and in the second direction, their respective driving surfaces 58, 59 remaining away from the medical instrument 5 so as not to drive the medical instrument 5, until the driving members 54, 56 of the second driving module 50′ regain their original position (FIG. 4G).

The control unit 90 is preferably configured to control the cyclic repetition of the steps of FIGS. 4D to 4G, in such a way as to allow the lengthening of the displacement path of the medical instrument 5 along the axis X.

The control unit 90 is in particular configured so that, when the driving members 54, 56 are brought closer to one another, i.e. in the steps illustrated by FIGS. 3B, 4B, 4D and 4F, the clamping force exerted by the driving members 54, 56 does not exceed a given limit threshold. Preferably, the limit threshold is less than or equal to 30 N, thus reducing the risk of damaging the medical instrument 5, particularly when the medical instrument 5 is a catheter.

For this purpose, the control unit 90 includes, for each driving sub-device 80, 82, a control sub-unit 98 of said sub-device 80, 82. With reference to FIG. 5, this control sub-unit 98 comprises a module 100 for controlling the actuators 62, 63, 64, 65, 66, 67 in position, a module 102 for controlling the speed of the actuators 62, 63, 64, 65, 66, 67 and a module 104 for controlling the force of the actuators 62, 63, 64, 65, 66, 67.

The position control module 100 comprises a first input 110 for receiving a given position setpoint along each of the axes of displacement of the driving member 54, 56 driven by the sub-device 80, 82 (thus here along each of the axes X, Y and Z) and a second input 112 for receiving a measurement of the position of said driving member 54, 56 along each of its axes of displacement (thus here along each of the axes X, Y and Z). The position control module 100 is configured to generate, as a function of these inputs, for each of the axes of displacement of the driving member 54, 56, a position control signal suitable for making the measured position along said axis of displacement converge on the corresponding position setpoint. It moreover comprises, for each axis of displacement of the driving member 54, 56, a respective output 114, 116, 188 for transmitting to each actuator 62, 63, 64, 65, 66, 67 concerned (i.e. to each actuator acting on the displacement of the driving member along said axis of displacement) this position control signal. Thus, in the example shown, said outputs 114, 116, 118 comprise:
a first output 114 for transmitting, to each actuator 62, 63, 64, 65, 66, 67 concerned, a position control signal suitable for making the position measured along the axis X converge on the position setpoint along the axis X,
a second output 116 for transmitting, to each actuator 62, 63, 64, 65, 66, 67 concerned, a position control signal suitable for making the position measured along the axis Y converge on the position setpoint along the axis Y, and
a third output 118 for transmitting, to each actuator 62, 63, 64, 65, 66, 67 concerned, a position control signal suitable for making the position measured along the axis Z converge on the position setpoint along the axis Z.

The speed control module 102 comprises a first input 120 for receiving a setpoint of speed along the clamping axis Y and a second input 122 for receiving a measurement of the speed of the driving member 54, 56 driven by the sub-device 80, 82 along the clamping axis Y. The speed control module 102 is configured to generate, as a function of these inputs, a speed control signal suitable for making the measured speed converge on the speed setpoint. It moreover comprises an output 124 to transmit to each actuator 62, 63, 64, 65, 66, 67 concerned (i.e. to each actuator acting on the displacement of the driving member along the clamping axis Y) this speed control signal.

The force control module 104 comprises a first input 130 for receiving a given force setpoint along the clamping axis Y and a second input 132 for receiving a measurement of the force exerted by the medical instrument 5 on the driving member 54, 56 driven by the sub-device 80, 82 along the clamping axis Y. The force control module 104 is configured to generate, as a function of these inputs, a force control signal suitable for making the measured force converge on the force setpoint. It moreover comprises an output 134 to transmit to each actuator 62, 63, 64, 65, 66, 67 concerned (i.e. to each actuator acting on the displacement of the driving member along the clamping axis Y) this force control signal. The force setpoint is strictly less than the limit threshold. It is for example less than 90% of the limit threshold, preferably between 80 and 90% of the limit threshold.

The control sub-unit 98 also comprises a switch 140 to switch the control of the clamping of the actuator 62, 63, 64, 65, 66, 67 between the position control, the speed control and the force control. For this purpose, the switch 140 comprises a first input 142 for receiving the position control signal, a second input 143 for receiving the speed control signal, and a third input 144 for receiving the force control signal. It moreover comprises an output 145 for transmitting a clamping control signal to each actuator 62, 63, 64, 65, 66, 67 concerned (i.e. to each actuator acting on the displacement of the driving member along the clamping axis Y). The switch 140 further comprises a fourth input 146 for receiving a measurement of the force exerted by the medical instrument 5 on said driving member 54, 56 along the clamping axis Y and a fifth input 147 for receiving an item of information about the current phase of displacement along the clamping axis Y (driving members 54, 56 moved closer to or apart from one another). The switch 140 is configured to connect the output 145 selectively to the first, second or third input 142, 143, 144 as a function of the fourth and fifth input 146, 147. In particular, the switch 140 is configured to switch between three configurations:
a first configuration in which it connects the output 145 to the first input 142, i.e. it transmits, as clamping signal, the position control signal to the actuators 62, 63, 64, 65, 66, 67 concerned, when the fifth input 147 indicates that the current phase of displacement along the clamping axis Y is a phase of moving apart of the driving members 54, 56 (i.e. typically in the steps illustrated by FIGS. 3D, 4D and 4F), whatever the value of the fourth input 146;
a second configuration in which it connects the output 145 to the second input 143, i.e. it transmits, as clamping signal, the speed control signal to the actuators 62, 63, 64, 65, 66, 67 concerned, when the fifth input 147 indicates that the current phase of displacement along the clamping axis Y is a phase of moving the driving members 54, 56 closer to one another, and that the measured clamping force received over the fourth input 146 is less than or equal to a given switching threshold; and
a third configuration in which it connects the output 145 to the third input 144, i.e. it transmits, as clamping signal, the force control signal to the actuators 62, 63, 64, 65, 66, 67 concerned, when the fifth input 147 indicates that the current phase of displacement along the clamping axis Y is a phase of moving the driving members 54, 56 closer to one another and that the measured clamping force received over the fourth input 146 is strictly greater than the given switching threshold.

The given switching threshold is strictly less than the force setpoint.

The clamping signal typically consists of an electrical signal controlling, for each actuator 62, 63, 64, 65, 66, 67 in question, the switching of the power switches of a current supply member of said actuator 62, 63, 64, 65, 66, 67.

The control sub-unit 98 further comprises a speed setpoint generator 150. This generator 150 comprises an input 152 for receiving a measurement of the position of the driving member 54, 56 driven by the sub-device 80, 82 along the clamping axis Y and an output 154 for transmitting the speed setpoint to the speed control module 102. The generator 150 is configured to compare the measured position to a given position threshold and to impart to the speed setpoint a first given value when the measured position is less than or equal to the position threshold and a second given value, strictly less than the first value, when the measured position is strictly greater than the position threshold.

The second value of the speed setpoint is preferably less than 90% of the first value, for example less than 60%, advantageously between 60 and 40% of the first value. Moreover, the second value is preferably less than the displacement speed reachable by the driving member 54, 56 during its displacement from its original position to the position threshold. The position threshold is such that when the measured position is equal to said position threshold the driving member 54, 56 is not in contact with the medical instrument 5. The position threshold is preferably obtained based on the maximum diameter of the different medical instruments that the driving member 54, 56 is liable to handle. Specifically, knowing the maximum diameter of the medical instrument 5 that the driving member 54, 56 can receive, it is possible to determine that before a given position it is not possible for the driving member 54, 56 to come into contact with the medical instrument 5. This in particular makes it possible to dispense with the measurement of the distance separating the driving member 54, 56 and the medical instrument 5, a measurement which can require a visual inspection device.

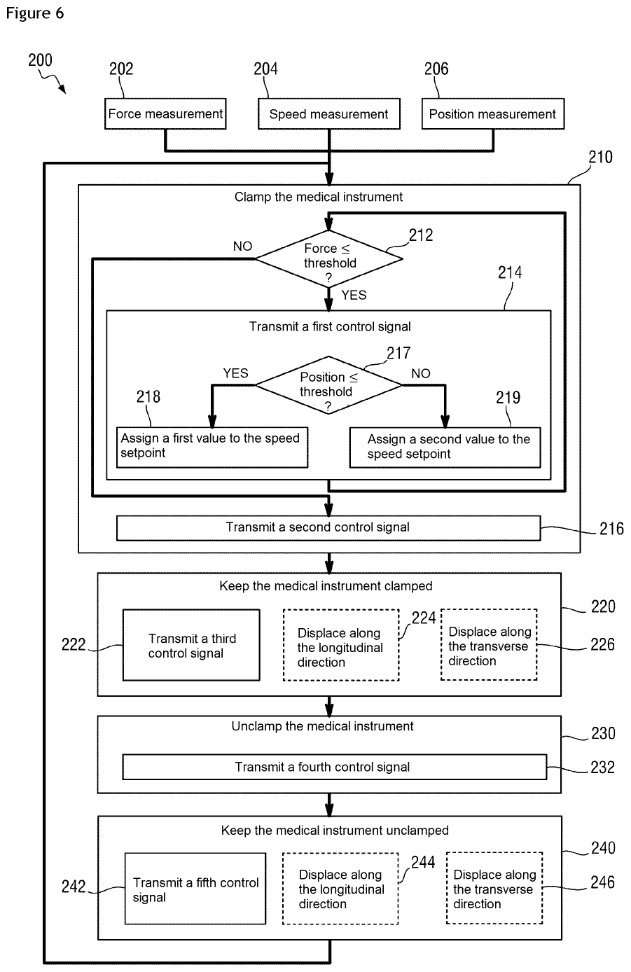

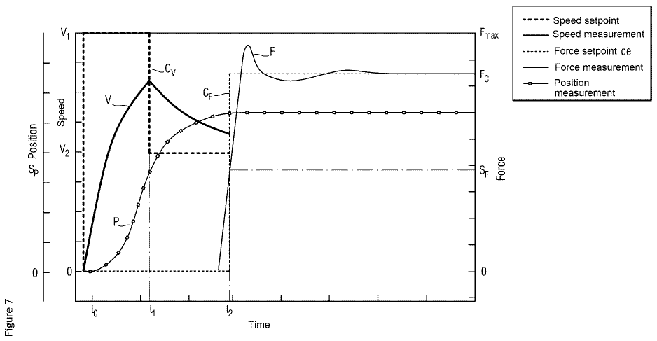

The method 200 implemented by the control unit 90 will now be described, with reference to FIGS. 6 and 7. For the sake of simplicity, the given description here relates to the control of the sole actuators 62, 63, 64 of one and the same driving sub-device 80 driving a sole driving member 54. Those skilled in the art will easily be able to deduce therefrom the control of the other actuators.

This method 200 further comprises a force measuring step 202, a speed measuring steep 204 and a position measuring step 206.

The force measuring step 202 comprises the measuring of the force exerted by the medical instrument 5 on the driving member 54 along the clamping axis Y. This force is typically deduced, as described above, from the supply power of the actuators 62, 63, 64. In a variant, this force is measured directly by a stress sensor.

The speed measuring step 204 comprises the measuring of the displacement speed of the driving member 54 along the clamping axis Y. This speed is typically deduced, as described above, from the current speed of at least one of the actuators 62, 63, 64. For example, the speed of displacement of the driving member 54 along the clamping axis Y is deduced from the current speed of the actuator 63 (i.e. of the actuator contributing mostly or exclusively to the displacement of the driving member 54 along the axis Y).

The position measuring step 206 comprises the measuring of the position of the driving member 54 along the clamping axis Y. This position is typically deduced, as described above, from the current position of at least one of the actuators 62, 63, 64. For example, the position of the driving member 54 along the clamping axis Y is deduced from the current position of the actuator 63 (i.e. of the actuator contributing mostly or exclusively to the displacement of the driving member 54 along the axis Y).

These steps 202, 204, 206 are repeated throughout the implementation of the method 200, at a given sampling frequency, optionally variable but preferably constant.

The method 200 then comprises a step 210 of clamping the medical instrument 5. During this step 210, the control unit 90 controls the actuators 62, 63, 64 such as to bring the driving member 54 closer to the driving member 56. This step 210 is typically implemented during the steps illustrated by FIGS. 3B, 4B, 4D and 4F.

The step 210 first of all comprises the comparing 212 of the measured force to the switching threshold. If the measured force is less than or equal to said threshold, this comparing 212 is followed by the transmitting 214 to the actuators 62, 63, 64 of a first control signal. If the measured force is strictly greater than said threshold, this comparing 212 is followed by the transmitting 216 to the actuators 62, 63, 64 of a second control signal. During the transmitting step 214, the switch 140 is in the second configuration: the first control signal therefore consists of the speed control signal. The first control signal is thus suitable for making the measured displacement speed converge on the speed setpoint supplied by the generator 150.

This transmitting step 214 comprises the comparing 217 of the measured position of the driving member 54 along the axis Y with the position threshold. If the measured position is less than or equal to said threshold, this comparing 217 is followed by the assigning 218 of the first value to the speed setpoint; in other words, the generator 150 confers the first value on the speed setpoint. If the measured position is strictly greater than said threshold, this comparing 217 is followed by the assigning 219 of the second value to the speed setpoint; in other words, the generator 150 confers the second value on the speed setpoint.

After the implementation of the transmitting step 214, the method 200 returns to the comparing of forces 212.

During the transmitting step 216, the switch 140 is in the third configuration: the second control signal therefore consists of the force control signal. The second control signal is thus suitable for making the measured force converge on the force setpoint.

The time-domain implementation of this clamping step 210 is illustrated by FIG. 7. The step 210 starts at a time t₀. At this time, the driving member 54 is still in its initial position along the axis Y; the measured position P has thus not yet reached the position threshold S_{P}. In addition, the driving member 54 is still distant from the medical instrument 5; the measured force F is therefore zero. It is therefore the first control law that is transmitted, with a speed setpoint C_{V} at the first value V₁. The measured speed V therefore gradually increases, tending to converge on the speed setpoint C_{V}.

Nonetheless, at the time t₁, here even before the speed V has reached the setpoint C_{V}, the measured position P reaches the threshold S_{P}. The driving member 54 is then still distant from the medical instrument 5, such that the measured force F is still zero. The first control law thus remains transmitted, but the value of the speed setpoint C_{V} is reduced to the second value V₂. Since here this is less than the value reached by the measured speed V at the time t₁, the speed V gradually decreases in such a way as to tend toward the new value of the speed setpoint C_{V}. The braking of the driving member 54 is thus begun.

It is during this deceleration phase that the driving member 54 comes into contact with the medical instrument 5, which causes an increase in the measured force F which then reaches the switching threshold S_{F} at a time t₂. This has the effect of stopping the transmission of the first control law to the actuators 62, 63, 64, and it is then the second control law that is transmitted, with a force setpoint CF equal to a value F_{C} which, as can be seen on this FIG. 7, is strictly less than a limit threshold Fₘₐₓ that one does not wish to exceed. Under the effect of this new control law, the measured force F briefly exceeds the value F_{C} but without crossing the threshold Fₘₐₓ then stabilizes at the force setpoint value C_{F}.

The implementation of the step 210 thus allows a rapid displacement of the driving member along the clamping axis Y, without crossing the limit threshold Fₘₐₓ.

Returning to FIG. 6, the step 210 is followed by a step 220 of keeping the medical instrument 5 clamped, during which the driving member 54 is substantially immobilized along the axis Y. This step 220 is typically implemented during the steps illustrated in FIGS. 3C, 4C, 4E and 4G.

This step 220 comprises the transmitting 222 to the actuators 62, 63, 64 of a third control signal suitable for keeping the measured force substantially equal to the force setpoint. For this purpose, the switch 140 is typically kept in its third configuration during this step 220.

Optionally, the step 220 also comprises the displacement 224 of the driving member 54 along the longitudinal axis X and/or the displacement 226 of the driving member 54 along the transverse axis Z. This displacement is typically obtained by superimposing on the third control signal a signal controlling the position along the axis X and/or along the axis Z supplied by the position control module 100.

The step 220 is itself followed by a step 230 of unclamping of the medical instrument 5. In this step 220, the control unit 90 controls the actuators 62, 63, 64 such as to move the driving member 54 apart from the driving member 56. This step 220 is typically implemented during the steps illustrated by FIGS. 3D, 4D and 4F.

The step 230 comprises the transmitting 232 of a fourth control signal to the actuators 62, 63, 64. During the step 230, the switch 140 is informed via its fifth input 147 that it is in a phase of moving apart of the driving members 54, 56 and is therefore switched into its first configuration. Thus, the fourth control signal consists of the position control signal. In other words, the fourth control signal is suitable for making the measured position converge on the position setpoint.

The step 230 is itself followed by a step 240 of keeping the medical instrument 5 unclamped, during which the driving member 54 is substantially immobilized along the axis Y. This step 240 is typically implemented during the steps illustrated on FIGS. 3E, 4E and 4G.

This step 240 comprises the transmitting 242 to the actuators 62, 63, 64 of a fifth control signal suitable for keeping the measured position along the axis Y substantially equal to the position setpoint. For this purpose, the switch 140 is typically kept in its first configuration during this step 240.

Optionally, the step 240 also comprises the displacing 244 of the driving member 54 along the longitudinal axis X and/or the displacing 246 of the driving member 54 along the transverse axis Z. This displacement is typically obtained by superimposing on the fifth control signal a signal controlling the position along the axis X and/or along the axis Z supplied by the position control module 100.

After the step 240, the method 200 finally returns to the step 210, the steps 210, 220, 230, 240 being thus repeated cyclically one after another, as described above in relation to FIGS. 3A to 3E and 4A to 4G.

Thus, owing to the exemplary embodiment described above, it is possible to displace the driving members 54, 56 quickly along the clamping axis Y, while avoiding the force exerted by the driving members 54, 56 on the medical instrument 5 along the axis Y exceeding the limit threshold Fmax, which makes it possible to prevent damage to the medical instrument 5. Furthermore, the clamping force is maintained and controlled during the step 220 of keeping the medical instrument 5 clamped, which here again makes it possible to avoid damage to the medical instrument 5 and further ensures the driving of the medical instrument 5 by the driving members 54, 56 without slipping.

According to an additional exemplary embodiment, the driving members are rollers which are rotary about the axis Z, the rotation of said rollers about the axis Z making it possible to translationally drive the medical instrument 5 along the axis X. Furthermore, these rollers are translationally movable along the clamping axis Y in order to clamp or release the medical instrument 5. Moreover, the rollers may be movable in translation along the axis Z in order to rotationally drive the medical instrument 5 about the axis X. Such a solution for the driving members is for example described in the patent application filed on 26 Apr. 2022 under number FR2203874. The management of the clamping described previously when the driving members are pad holders to which single-use pads are attached can be applied in a similar way when the driving members are rotary rollers.

1. A method for controlling an actuator of a robotic system for driving, in at least one of translation or rotation, an elongate flexible medical instrument, the robotic system comprising a driving member able to come into contact with the elongate flexible medical instrument and kinematically linked to the actuator such that the latter controls the movement of said driving member along a so-called main axis, the control method being implemented by a data processing unit and comprising the following steps:
measuring a speed of movement of the driving member along the main axis,
measuring a force exerted by the elongate flexible medical instrument on the driving member along the main axis, and
displacing the driving member along the main axis, in a first direction, with:
transmitting to the actuator, a primary control signal suitable for making the speed of movement converge on a given speed setpoint as long as the force is below a non-zero given force threshold, and
transmitting to the actuator, a secondary control signal suitable for making the force converge on a given force setpoint when the measured force exceeds said force threshold.

2. The control method as claimed in claim 1, wherein the main axis consists in a clamping axis of the elongate flexible medical instrument, the first direction being oriented toward the elongate flexible medical instrument.

3. The control method as claimed in claim 1, comprising the following additional steps:
measuring measured position of the driving member along the main axis, and
displacing the driving member along the main axis in a second direction opposite to the first direction, with transmitting to the actuator a tertiary control signal suitable for making the measured position converge on a given position setpoint.

4. The control method as claimed in claim 3, comprising between the steps of displacing in the first direction and in the second direction an additional step of immobilizing the driving member along the main axis during which there is transmitted to the actuator a quaternary control signal suitable for keeping the measured force substantially equal to the force setpoint.

5. The control method as claimed in claim 4, comprising, during the step of immobilizing the driving member along the main axis, displacing the driving member along at least one axis orthogonal to the main axis.

6. The control method as claimed in claim 3, wherein the steps of displacing in the first direction and in the second direction are implemented one after the other, in a cyclic repeated manner.

7. The control method as claimed in claim 3, wherein the measured position is deduced from a current position of the actuator.

8. The control method as claimed in claim 1, comprising an additional step of measuring a measured position of the driving member along the main axis, the speed setpoint having a first value as long as the measured position is below a given position threshold and a second value, less than the first value, when the measured position is above said position threshold.

9. The control method as claimed in claim 8, wherein the position threshold is such that when the measured position is equal to said position threshold the driving member is not in contact with the elongate flexible medical instrument.

10. The control method as claimed in claim 1, wherein the force setpoint is greater than the force threshold.

11. The control method as claimed in claim 1, wherein the speed of movement is deduced from a current speed of the actuator.

12. The control method as claimed in claim 1, wherein the force is deduced from a current supply power of the actuator.

13. The control method as claimed in claim 1, wherein the elongate flexible medical instrument consists of a catheter or a catheter guide.

14. A robotic system for driving an elongate flexible medical instrument, said robotic system comprising a frame, a driving member able to come into contact with the elongate flexible medical instrument, an actuator kinematically linked to the driving member such as to control the movement of said driving member in relation to the frame along a main axis, and a data processing unit for implementing the method as claimed in any claim 1.

15. A computer program product comprising code instructions for implementing the control method as claimed in claim 1 when said computer program product is executed by a processor of a data processing unit of a robotic system for driving an elongate flexible medical instrument.
